(19) United States
(12) Patent Application Publication
(10) Pub. No.: US 2026/0183234 A1
(43) Pub. Date: Jul. 2, 2026

(54) CELL-ADHESIVE POLYAMINO COMPOUNDS AND MEDICAL HYDROGELS FORMED THEREFROM

(71) Applicant: Boston Scientific Scimed, Inc., Maple Grove, MN (US)

(72) Inventors: Yen-Hao Hsu, Shrewsbury, MA (US); Heather Cirka, Hudson, MA (US); Nishant Khattar, White Bear Township, MN (US)

(73) Assignee: Boston Scientific Scimed, Inc., Maple Grove, MN (US)

(21) Appl. No.: 19/429,424

(22) Filed: Dec. 22, 2025

Related U.S. Application Data
(60) Provisional application No. 63/740,039, filed on Dec. 30, 2024.

(51) Int. Cl. A61K 9/06; A61K 47/34; A61K 47/42

(52) U.S. Cl. CPC A61K 9/06; A61K 47/34; A61K 47/42

(57) ABSTRACT

In some aspects, the present disclosure provides systems for forming hydrogels that comprise a cell-adhesive polyamino compound and a reactive multi-arm polymer that comprises a plurality of hydrophilic polymer arms having reactive end groups that are reactive with amino groups of the cell-adhesive polyamino compound. In other aspects, the present disclosure provides crosslinked reaction products of a cell-adhesive polyamino compound and a reactive multi-arm polymer that comprises a plurality of hydrophilic polymer arms having reactive end groups that are reactive with amino groups of the cell-adhesive polyamino compound. In other aspects, the present disclosure pertains to methods of treatment using such systems and crosslinked products.

### CROSS-REFERENCE TO RELATED APPLICATIONS

This application claims the benefit of U.S. Provisional Patent Application Ser. No. 63/740,039 filed on Dec. 30, 2024, the disclosure of which is incorporated herein by reference.

### FIELD

The present disclosure relates to cell-adhesive polyamino compounds, to hydrogels formed from cell-adhesive polyamino compounds, and to methods of making and using cell-adhesive polyamino compounds. The compounds of the present disclosure are useful, for example, in forming hydrogels for various biomedical applications.

### BACKGROUND

Bioresorbable hydrogels with rapid crosslinking reaction rates in vivo, known by the trade name of SpaceOAR®, have become a prominent biomaterial and obtained clinical success in creating the space between prostate and rectum, tremendously improving patient safety during the cancer therapies. SpaceOAR® is based on a multi-arm polyethylene glycol (PEG) polymer with a polyol core functionalized with succinimidyl glutarate (SG) as reactive end groups which further react with trilysine to form crosslinks. A further improvement based on this application is that some of 8-Arm PEG branches are functionalized with 2,3,5-triiodobenzamide (TIB) groups, replacing part of the SG groups, in order to provide intrinsic radiopacity to the hydrogels themselves for CT-visibility. This hydrogel is known by the trade name of SpaceOAR Vue®. The hydrogels break down in-vivo over the course of about 6-9 months. The breakdown occurs primarily through the hydrolysis of ester linkages in the glutarate groups.

The present disclosure provides implantable hydrogel alternatives to SpaceOAR® and SpaceOAR Vue®, which promote cell adhesion and tissue growth proximate the hydrogel, thereby expanding the range of medical applications for the implantable hydrogel alternatives.

### SUMMARY

In some aspects, the present disclosure provides systems for forming hydrogels that comprise a cell-adhesive polyamino compound and a reactive multi-arm polymer that comprises a plurality of hydrophilic polymer arms having reactive end groups that are reactive with amino groups of the cell-adhesive polyamino compound.

In some embodiments, the cell-adhesive polyamino compound comprises a polyamino moiety that is linked to a cell-adhesive peptide moiety by an amide group. In some of these embodiments, the polyamino moiety comprises a plurality of —(CH₂)ₓ—NH₂ groups where x is 0, 1, 2 3, 4, 5 or 6 and/or the polyamino moiety is a polyamino peptide moiety and/or the polyamino moiety comprises two or more amino acid residues selected from residues of lysine, ornithine, and combinations thereof.

In some embodiments, which can be used in conjunction with any of the above aspects and embodiments, the cell-adhesive peptide moiety comprises one or more Arg-Gly-Asp sequences.

In some embodiments, which can be used in conjunction with any of the above aspects and embodiments, the cell-adhesive polyamino compound comprises a covalently attached iodine atom.

In some embodiments, which can be used in conjunction with any of the above aspects and embodiments, the polyamino moiety comprises a residue of a carboxylic-acid-containing iodinated molecule.

In some embodiments, which can be used in conjunction with any of the above aspects and embodiments, the cell-adhesive polyamino compound comprises an iodinated amino acid residue.

In some embodiments, which can be used in conjunction with any of the above aspects and embodiments, the hydrophilic polymer arms comprise polymer segments that comprise one or more hydrophilic monomers selected from ethylene oxide, N-vinyl pyrrolidone, oxazoline monomers, hydroxyethyl acrylate, and hydroxyethyl methacrylate.

In some embodiments, which can be used in conjunction with any of the above aspects and embodiments, the reactive end groups are linked to the hydrophilic polymer arms by a hydrolysable ester.

In some embodiments, which can be used in conjunction with any of the above aspects and embodiments, the reactive multi-arm polymer does not comprise a hydrolysable ester group.

In some embodiments, which can be used in conjunction with any of the above aspects and embodiments, the reactive end groups are electrophilic groups.

In some embodiments, which can be used in conjunction with any of the above aspects and embodiments, the system comprises a first composition that comprises the cell-adhesive polyamino compound, a second composition that comprises the reactive multi-arm polymer, and an optional accelerant composition. In some of these embodiments, the first composition is provided in a syringe barrel, the second composition is provided in a vial, and the accelerant composition is provided in a syringe barrel.

In other aspects, the present disclosure provides crosslinked products of a cell-adhesive polyamino compound in accordance with any of the above aspects and embodiments and a reactive multi-arm polymer in accordance with any of the above aspects and embodiments.

In some embodiments, the crosslinked product does not contain hydrolysable ester groups.

In other aspects, the present disclosure provides injectable suspensions comprising particles of a crosslinked product in accordance with the above aspects and embodiments.

In other aspects, the present disclosure pertains to methods of treatment comprising administering to a subject a mixture that comprises a cell-adhesive polyamino compound in accordance with any of the above aspects and embodiments and a reactive multi-arm polymer in accordance with any of the above aspects and embodiments under conditions such that the cell-adhesive polyamino compound and the reactive multi-arm polymer cross-link after administration.

In other aspects, the present disclosure pertains to methods of treatment comprising administering to a subject a crosslinked product of a cell-adhesive polyamino compound in accordance with any of the above aspects and embodiments and a reactive multi-arm polymer in accordance with any of the above aspects and embodiments.

The present disclosure is advantageous as it provides implantable hydrogels that promote cell adhesion and tissue growth upon implantation, providing, for example, resistance to migration after implantation of the implantable hydrogels.

The above and other aspects, embodiments, features and benefits of the present disclosure will be readily apparent from the following detailed description.

### BRIEF DESCRIPTION OF THE DRAWINGS

FIG. 1 schematically illustrates a crosslinking reaction, in accordance with an embodiment of the present invention.

FIG. 2 schematically illustrates cell adhesion, cell proliferation, and tissue growth associated with a crosslinked hydrogel implant, in accordance with an embodiment of the present invention.

FIGS. 3A-B schematically illustrate delivery of a crosslinked hydrogel implant into a ureteral wall, in accordance with an embodiment of the present invention.

FIG. 4 illustrates a delivery device, in accordance with an embodiment of the present disclosure.

FIG. 5 illustrates a delivery device, in accordance with another embodiment of the present disclosure.

### DETAILED DESCRIPTION

In various aspects, the present disclosure provides cell-adhesive polyamino compounds that comprise a polyamino moiety that is linked to a cell-adhesive peptide moiety. In some of these embodiments, the polyamino moiety is linked to the cell-adhesive peptide moiety through an amide group.

The cell-adhesive polyamino compounds of the present disclosure comprise a polyamino moiety having a plurality of (e.g., two, three, four, five, six, seven, eight, nine, ten or more) amino groups. For example, the polyamino moiety may comprises a plurality of (e.g., two, three, four, five, six, seven, eight, nine, ten or more) —(CH₂)ₓ—NH₂ groups where x is 0, 1, 2 3, 4, 5 or 6. In some embodiments, the polyamino moiety may comprise a plurality of —(CH₂)ₓ—NH₂ groups disposed along a polymeric moiety (defined herein as a moiety comprising 2, 3, 4, 5, 6, 7, 8, 9, 10 or more monomer residues). In some of these embodiments, the polymeric moiety may be selected from a polyamide moiety, a polyalkylene moiety, or a polysaccharide moiety, among others.

In some embodiments, the polyamino moieties are polyamino peptide moieties. Polyamino peptide moieties of the present disclosure typically contain amino acid sequences that have between 3 and 20 residues of one or more types of amino acids that have primary-amine-containing side groups, such as, for example, residues of lysine and/or ornithine. Examples of polyamino peptide moieties thus include polyamino peptide moieties that contain polylysine residues such as residues of dilysine, trilysine, tetralysine, pentalysine, etc., polyornithine residues such as residues of diornithine, triornithine, tetraornithine, pentaornithine, etc., and poly(lysine-co-ornithine) residues.

Cell-adhesive peptide moieties of the present disclosure typically contain amino acid sequences that have between 3 and 20 amino acid residues. Cell-adhesive peptide moieties of the present disclosure include cell-adhesive peptide moieties that comprise one or more RGD amino acid sequences, cell-adhesive peptide moieties that comprise one or more IKVAV sequences, cell-adhesive peptide moieties that comprise one or more YIGSR sequences, cell-adhesive peptide moieties that comprise one or more DGEA sequences, cell-adhesive peptide moieties that comprise one or more GFOGER sequences, cell-adhesive peptide moieties that comprise one or more sequences, cell-adhesive peptide moieties that comprise one or more GFPGER sequences, cell-adhesive peptide moieties that comprise one or more PHSRN sequences, cell-adhesive peptide moieties that comprise one or more REDV sequences, cell-adhesive peptide moieties that comprise one or more LDV sequences, cell-adhesive peptide moieties that comprise one or more KQAGDV sequences, cell-adhesive peptide moieties that comprise one or more VSWRAPTA sequences, and cell-adhesive peptide moieties that comprise one or more VFDNFVLK sequences, among others.

With regard to cell-adhesive peptide moieties that comprise one or more RGD amino acid sequences, it is noted that biomaterials incorporating bound RGD (Arg-Gly-Asp) peptide sequences have found success not only in academic research but also in clinical trials such as inducing cell adhesion, promoting tissue regeneration/growth, drug delivery, and cancer treatment. See, Javid, H., et al. (2024). RGD peptide in cancer targeting: Benefits, challenges, solutions, and possible integrin-RGD interactions. Cancer Medicine, 13(2), e6800 and Wandel, M. B., et al. (2021). Concomitant control of mechanical properties and degradation in resorbable elastomer-like materials using stereochemistry and stoichiometry for soft tissue engineering. Nature Communications, 12(1), 446. RGD sequences may be included in the hydrogels of the present disclosure to draw fibroblasts to the implant site and encourage fibroblast attachment/spreading and new tissue synthesis. See, e.g., Rajagopalan, Padmavathy, et al. Direct comparison of the spread area, contractility, and migration of balb/c 3T3 fibroblasts adhered to fibronectin- and RGD-modified substrata. Biophysical journal 87.4 (2004): 2818-2827.

In some embodiments, the cell-adhesive polyamino compounds of the present disclosure may be formed by a coupling reaction between a cell-adhesive peptide and a polyamino compound.

Examples of polyamino compounds include compounds that comprise a plurality of (e.g., two, three, four, five, six, seven, eight, nine, ten or more) amino groups. For example, the polyamino compound may comprises a plurality of (e.g., two, three, four, five, six, seven, eight, nine, ten or more) —(CH₂)ₓ—NH₂ groups where x is 0, 1, 2 3, 4, 5 or 6. In some of these embodiments, the polyamino compound may comprises a plurality of —(CH₂) «—NH₂ groups disposed along a polymeric moiety (defined herein as a moiety comprising 2, 3, 4, 5, 6, 7, 8, 9, 10 or more monomer residues). In some embodiments, the polymeric moiety may be selected from a polyamide moiety, a polyalkylene moiety, or a polysaccharide moiety, among others.

Particular examples polyamino compounds include poly(amino acids) that comprise a plurality of primary-amine-containing side groups (e.g., 2, 3, 4, 5, 6, 7, 8, 9, 10 or more primary-amine-containing side groups), such as, for example, poly(amino acids) that comprise lysine and/or ornithine (e.g., polylysine compounds such as dilysine, trilysine, tetralysine, pentalysine, etc., polyornithine compounds such as diornithine, triornithine, tetraornithine, pentaornithine, etc., and poly(lysine-co-ornithine) compounds).

In some embodiments, the cell-adhesive polyamino compounds of the present disclosure may be formed by an amide coupling reaction between a cell-adhesive peptide and a polyamino peptide.

For example, the carboxylic acid group at the C-terminus of a polyamino peptide can be linked with the amino group at the N-terminus of a cell-adhesive peptide in an amide coupling reaction. Prior to reaction, the carboxylic acid group at the C-terminus of the cell-adhesive peptide and the amino group at the N-terminus of the polyamino peptide are provided with suitable protective groups. Moreover, any side chains of the cell-adhesive peptide and the polyamino peptide that can interfere with the amide coupling reaction between the amino group at the N-terminus of the cell-adhesive peptide and the carboxylic acid group at the C-terminus of the polyamino peptide are also provided with suitable protective groups.

In a particular example where the carboxylic acid group at the C-terminus of trilysine (Lys-Lys-Lys),

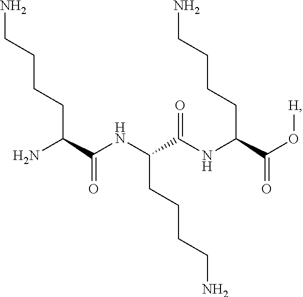

is reacted with the amino group at the N-terminus of RGD peptide, Arg-Gly-Asp,

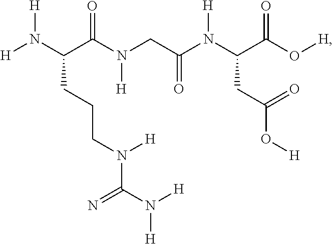

to form Lys-Lys-Lys-Arg-Gly-Asp,

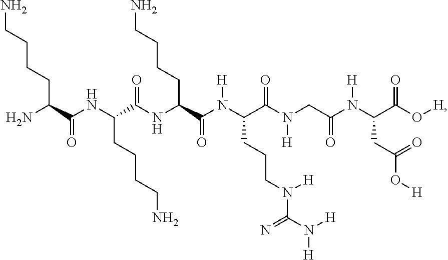

the amino group at the N-terminus of the Lys-Lys-Lys, the primary amine groups in the lysine side chains of the Lys-Lys-Lys, the guanidine group in the arginine side chain of the Arg-Gly-Asp, the carboxylic acid group at the C-terminus of the Arg-Gly-Asp, and the carboxylic acid group of the aspartic acid side chain of the Arg-Gly-Asp are protected.

Although a polyamino peptide, specifically trilysine is used in the preceding example, an analogous reaction can be performed with non-peptide, polyamino compounds that comprise a carboxylic acid group in addition to a plurality (two, three, four, five, six, seven, eight, nine, ten or more) amino groups. Particular examples of such non-peptide polyamino compounds include carboxylic-acid-terminated polyamines such as a carboxylic-acid-terminated poly(allyl amine), a carboxylic-acid-terminated poly(vinyl amine), or a carboxylic-acid-terminated chitosan, among others. As with the trilysine in the preceding example, the amine groups of the carboxylic-acid-terminated polyamines are protected during the coupling reaction.

Alternatively, the amino group at the N-terminus of a polyamino peptide can be linked in an amide coupling reaction with the carboxylic acid group at the C-terminus of a cell-adhesive peptide. Prior to reaction, the carboxylic acid group at the C-terminus of the polyamino peptide and the amino group at the N-terminus of the cell-adhesive peptide are provided with suitable protective groups. Moreover, any side chains of the cell-adhesive peptide and the polyamino peptide that could interfere with the amide coupling reaction between the amino group at the N-terminus of the polyamino peptide and the carboxylic acid group at the C-terminus of the cell-adhesive peptide are also provided with suitable protective groups.

In a particular example where the amino group at the N-terminus of Lys-Lys-Lys, is reacted with the carboxylic acid group at the C-terminus of Arg-Gly-Asp to form Arg-Gly-Asp-Lys-Lys-Lys, the guanidine group in the arginine side chain of the Arg-Gly-Asp, the primary amine groups in the lysine side chains of the Lys-Lys-Lys, the carboxylic acid group at the C-terminus of the Lys-Lys-Lys, the carboxylic acid group in the aspartic acid side chain of the Arg-Gly-Asp are protected.

Although a polyamino peptide, specifically trilysine, is used in the preceding example, an analogous reaction can be performed with non-peptide, polyamino compounds that comprise three or more (three, four, five, six, seven, eight, nine, ten or more) amino groups in which all but one of the amino groups are protected during the coupling reaction. Particular examples of such non-peptide polyamino compounds include amino-terminated polyamines such as an amino-terminated poly(allyl amine), an amino-terminated poly(vinyl amine), or an amino-terminated chitosan, among others.

The preceding amide coupling reactions are typically performed in the presence of a suitable amide coupling agent. Examples of suitable amide coupling agents include carbodiimide coupling agents, such as N,N′-dicyclohexylcarbodiimide (DCC), 1-ethyl-3-(3-dimethyl propyl) carbodiimide (EDC), 1,3-diisopropylcarbodiimide (DIC), N-hydroxybenzotriazole (HOBt), (benzotriazol-1-yloxy)tris(dimethylamino)phosphonium hexafluorophosphate (BOP reagent), and 2-(1H-Benzotriazole-1-yl)-1,1,3,3-tetramethylaminium tetrafluoroborate (TBTU), among others.

In some embodiments, the cell-adhesive polyamino compounds of the present disclosure may be formed from individual amino acids using known methods of peptide synthesis including liquid phase synthesis schemes and solid phase peptide synthesis schemes (e.g., Wang's method). For instance, in one example of a solid phase peptide synthesis scheme (a) a first amino acid (with a protected amino group) is covalently linked to a solid support (e.g., a resin) at its C-terminus, (b) the amino protecting group is then removed, exposing the free amino group and making it available for the next coupling reaction, (c) a new protected amino acid is added to the growing peptide chain, typically using a suitable amide coupling agent, forming a peptide bond with the deprotected amino group, (d) excess reagents and byproducts are then removed by washing the resin, (e) steps (b)-(d) are repeated until the desired peptide sequence is constructed, and (f) the completed peptide is cleaved from the resin, usually with chemical reagents that also remove any remaining protecting groups.

As above, depending on the nature of the side chains, it will be desirable to employ protective groups to prevent unwanted side reactions from occurring during the synthesis process. For example, it may be desirable to protect amine, carboxylic acid, alcohol, or thiol side chain groups during the synthesis process.

Examples of protective groups for use in conjunction with the present disclosure include tert-butoxycarbonyl (Boc) groups, carboxybenzyl (Cbz) or (Z) groups, trifluoroacetyl (TFA) groups, 6-nitroveratryloxycarbonyl (Nvoc) groups, 9-fluorenylmethoxycarbonyl (Fmoc) groups, 2,2,4,6,7-pentamethyldihydrobenzofuran-5-sulfonyl (Pbf) groups, allyloxycarbonyl (Alloc) groups, trityl (Trt) groups, t-butyl (t-Bu) groups, benzyl ester (β-benzyl) groups, O-benzyl groups, S-ethylsulfonyl groups, 2,4-dimethoxybenzyl (Dmb), tert-butyldimethylsilyl (TBDMS), allyl, ortho-nitrobenzyl (PNB), para-methylbenzyl (Meb), and acetamidomethyl (Acm) groups, among others.

For example, a common side chain protective group used for lysine is the tert-butoxycarbonyl (Boc) group, which is often used in combination with an Fmoc protecting group on the alpha-amino group. The Fmoc group is base-labile, allowing for easy deprotection during peptide chain elongation, while the Boc group on the lysine side chain is acid-labile, enabling deprotection under acidic conditions, frequently in conjunction with cleavage from the resin at the end of solid phase peptide synthesis, using trifluoroacetic acid (TFA). An example of a side chain protective group for arginine is 2,2,4,6,7-pentamethyldihydrobenzofuran-5-sulfonyl (Pbf) group, which protects the guanidine side chain, is acid labile, and can also be removed with TFA. An example of a side chain protective group for aspartic acid is a tert-butyl (tBu) ester group, which protects the carboxylic acid side chain, is acid labile, and can be removed with TFA. Other protective groups for these and other amino acids are well-known in the peptide synthesis art.

In some embodiments, the cell-adhesive polyamino compounds of the present disclosure have radiopacity. One way to introduce radiopacity is to include one or more iodinated amino acids in the cell-adhesive polyamino compounds. Examples of iodinated amino acids include amino acids that comprise an iodinated aromatic group. Examples of iodinated aromatic groups include iodo-phenyl groups and iodo-naphthyl groups. In particular embodiments, the iodinated amino acids include amino acids that comprise a hydroxy-iodo-aromatic group, such as a hydroxy-iodo-aromatic group or a hydroxy-iodo-naphthyl group. More particular examples of hydroxy-iodo-aromatic groups include hydroxy-iodo-aromatic groups selected from a mono-hydroxy-mono-iodo-aromatic group, a mono-hydroxy-di-iodo-aromatic group, a mono-hydroxy-tri-iodo-aromatic group, a mono-hydroxy-tetra-iodo-aromatic group, a di-hydroxy-mono-iodo-aromatic group, a di-hydroxy-di-iodo-aromatic group, a di-hydroxy-tri-iodo-aromatic group, a tri-hydroxy-mono-iodo-aromatic group, a tri-hydroxy-di-iodo-aromatic group, as previously indicated.

Specific examples of iodinated amino acids include the following: iodo-phenylalanine,

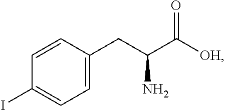 [SMILES: N[C@@H](Cc1ccc(I)cc1)C(=O)O]

which comprises a mono-iodo-aromatic group, monoiodotyrosine,

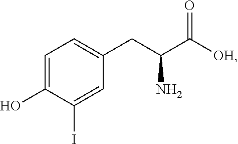 [SMILES: N[C@@H](Cc1ccc(O)c(I)c1)C(=O)O]

which comprises a mono-iodo-aromatic group, specifically, a mono-hydroxy-mono-iodo-aromatic group, diiodotyrosine,

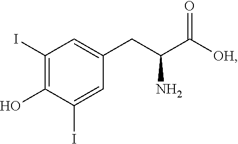 [SMILES: N[C@@H](Cc1cc(I)c(O)c(I)c1)C(=O)O]

which comprises a di-iodo-aromatic group, specifically a mono-hydroxy-di-iodo-aromatic group, diiodothyronine,

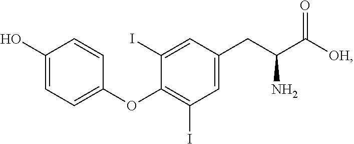 [SMILES: N[C@@H](Cc1cc(I)c(Oc2ccc(O)cc2)c(I)c1)C(=O)O]

which comprises a di-iodo-aromatic group and a hydroxy-aromatic group, triiodothyronine also known as T3,

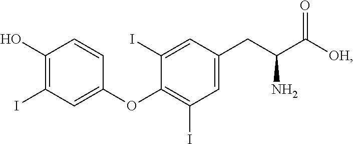 [SMILES: N[C@@H](Cc1cc(I)c(Oc2ccc(O)c(I)c2)c(I)c1)C(=O)O]

which comprises a di-iodo-aromatic group and a mono-hydroxy-mono-iodo-aromatic group, tetraiodothyronine also known as thyroxine or T4,

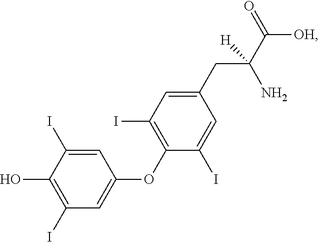 [SMILES: N[C@@H](Cc1cc(I)c(Oc2cc(I)c(O)c(I)c2)c(I)c1)C(=O)O]

which comprises a di-iodo-aromatic group and a mono-hydroxy-di-iodo-aromatic group, and 6-iodo-L-DOPA, which comprises a di-hydroxy-mono-iodo-aromatic group, among others.

Such iodinated amino acids may be included during or after synthesis of the above-described cell-adhesive polyamino compounds.

Another way to introduce radiopacity is to react a carboxylic-acid-containing iodinated molecule with the cell-adhesive polyamino compounds once formed.

For example, a carboxylic acid group of a carboxylic-acid-containing iodinated molecule may be reacted with the amino group at the N-terminus of a cell-adhesive polyamino compound in an amide coupling step. Analogous to the above procedures, protective groups are generally provided for any functional groups in the cell-adhesive polyamino compound (or the carboxylic-acid-containing iodinated molecule) that can interfere with the amide coupling reaction between the carboxylic acid group of the carboxylic-acid-containing iodinated molecule and the amino group at the N-terminus of the cell-adhesive polyamino compound. Also analogous to the above procedures, an amide coupling reagent is typically employed.

Examples of carboxylic-acid-containing iodinated compounds include those that comprise at least one iodinated aromatic group (e.g., a monocyclic or multicyclic aromatic structure that is substituted with one, two, three, four, five, six or more iodine atoms) and a carboxylic acid group. Specific examples of carboxylic-acid-containing iodinated molecule include the following, among many others: hydroxymethyl iodobenzoic acid,

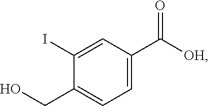 [SMILES: O=C(O)c1ccc(CO)c(I)c1]

hydroxymethyl diiodobenzoic acid,

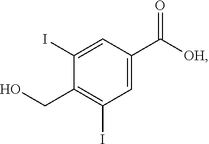 [SMILES: O=C(O)c1cc(I)c(CO)c(I)c1]

4, 4-bis(hydroxy-3, 5-diiodophenyl) pentanoic acid (IBHP),

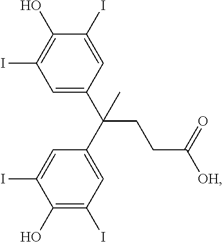 [SMILES: CC(CCC(=O)O)(c1cc(I)c(O)c(I)c1)c1cc(I)c(O)c(I)c1]

triiodobenzoic acid

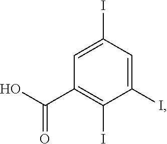 [SMILES: O=C(O)c1cc(I)cc(I)c1I]

diatrizoic acid,

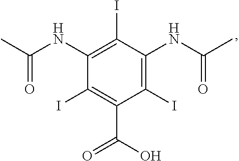 [SMILES: CC(=O)Nc1c(I)c(NC(C)=O)c(I)c(C(=O)O)c1I]

N-acetyl-3,5-diiodo-L-tyrosine,

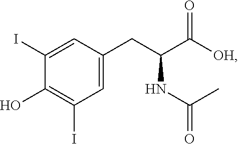 [SMILES: CC(=O)N[C@@H](Cc1cc(I)c(O)c(I)c1)C(=O)O]

N-acetyl-3-diiodo-L-tyrosine,

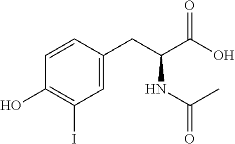 [SMILES: CC(=O)N[C@@H](Cc1ccc(O)c(I)c1)C(=O)O]

and N-acetyl-thyroxine,

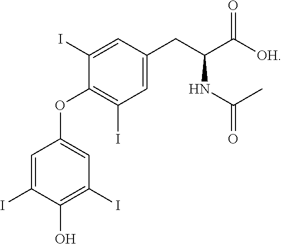 [SMILES: CC(=O)N[C@@H](Cc1cc(I)c(Oc2cc(I)c(O)c(I)c2)c(I)c1)C(=O)O]

Various carboxylic-acid-containing iodinated molecules, along with their CAS numbers, are listed in the following table:

| Name | CAS | # I atoms |
|---|---|---|
| 2-Iodobenzoic acid | 88-67-5 | 1 |
| 4-Iodobenzoic acid | 619-58-9 | 1 |
| 3-Iodobenzoic acid | 618-51-9 | 1 |
| 4-Iodopicolinic acid | 405939-79-9 | 1 |
| 3,5-Diiodobenzoic acid | 19094-48-5 | 2 |
| 3,4-Diiodobenzoic acid | 35674-20-5 | 2 |
| 3,5-Diiodo-4-(4-methoxyphenoxy)benzoic acid | 34043-77-1 | 2 |
| Diatrizoate | 117-96-4 | 3 |
| 2,3,5-Triiodobenzoic acid | 88-82-4 | 3 |
| Acetrizoic acid | 85-36-9 | 3 |
| Metrizoate | 1949-45-7 | 3 |
| 3,4,5-Triiodobenzoic acid | 2338-20-7 | 3 |
| Ioxitalamic acid | 28179-44-4 | 3 |
| Iothalamic acid | 2276-90-6 | 3 |
| 2,4,6-Triiodo-1,3,5-benzenetricarboxylic acid | 79211-41-9 | 3 |
| 2,4,6-Triiodobenzoic acid | 2012-31-9 | 3 |
| Benzoic acid, 3-[[2-Hydroxy-1- | 87932-11-4 | 3 |
| (hydroxymethyl)ethyl]amino]carbonyl]-5-[(2- | | |
| hydroxy-1-oxopropyl)amino]-2,4,6-triiodo-, | | |
| (S)-(9CI) | | |
| Ioseric acid | 51876-99-4 | 3 |
| 4-[4-(Acetyloxy)-3-iodophenoxy]-3,5- | 2260-084 | 3 |
| diiodobenzoic acid | | |
| Ioglicic acid | 49755-67-1 | 3 |
| 2,4,6-Triiodo-3-[(1-oxo-3,6,9,12,15- | 16024-67-2 | 3 |
| pentaoxahexadec-1-yl)amino]benzoic acid | | |
| 3-[[(1,1-Dimethylethoxy)carbonyl]amino]-2,4,6- | 2358047-48-8 | 3 |
| triiodobenzoic acid | | |
| 2,3,4,6-Tetraiodobenzoic acid | 71463-71-3 | 4 |
| Tetraform | 2055-97-2 | 4 |
| 2,3,5,6-Tetraiodo-1,4-benzenedicarboxylic acid | 7606-84-0 | 4 |
| N-Acetyl-O-(4-hydroxy-3,5-diiodophenyl)-3,5- | 26041-51-0 | 4 |
| diiodo-L-tyrosine | | |
| D-Tyrosine, N-[(1,1-dimethylethoxy)carbonyl]- | 89624-64-6 | 4 |
| O-(4-hydroxy-3,5-diiodophenyl)-3,5-diiodo- | | |
| (9CI, ACI) | | |
| 2,3,4,5,6-Pentaiodobenzoic acid | 64385-02-0 | 5 |
| Ioxaglic acid | 59017-64-0 | 6 |
| Ioglycamic acid | 2618-25-9 | 6 |
| Iocarmic acid | 10397-75-8 | 6 |

In other aspects of the present disclosure, crosslinked reaction products of (a) a cell-adhesive polyamino compound such as those described above and (b) a reactive multi-arm polymer that comprises a plurality of polymer arms that have reactive end groups that are reactive with the amino groups of the cell-adhesive polyamino compound, are provided.

In some embodiments, the crosslinked reaction products are hydrogels. As used herein, a “hydrogel,” which may also be referred to herein as a “crosslinked hydrogel,” is a crosslinked polymer that contains water or can absorb water but does not dissolve when placed in water.

In various embodiments, such crosslinked products are visible under fluoroscopy. In various embodiments, such crosslinked products have a radiopacity that is greater than 100 Hounsfield units (HU), beneficially anywhere ranging from 100 HU to 250 HU to 500 HU to 750 HU to 1000 HU to 2000 HU or more (in other words, ranging between any two of the preceding numerical values), for example, when measured on a bench-top micro CT system such as Xtreme CT from Scanco Medical (Wangen-Brüttisellen, Switzerland) or similar.

Such crosslinked reaction products may be formed in vivo (e.g., using a delivery device like that described below), or such crosslinked products may be formed ex vivo and subsequently administered to a subject. Such crosslinked products can be used in a wide variety of biomedical applications, including medical devices, implants, and pharmaceutical compositions.

In some embodiments, for example, where it is desired to form a bioresorbable hydrogel, the reactive multi-arm polymer comprises hydrolysable groups.

In some embodiments, for example, where it is desired to form a biostable hydrogel, the reactive multi-arm polymer does not comprise hydrolysable groups.

In various embodiments, the reactive end groups of the reactive multi-arm polymer and the amino groups of the cell-adhesive polyamino compound react with one another via an amide coupling reaction to form a crosslinked product

Reactive multi-arm polymers for use in the present disclosure include reactive multi-arm polymers that comprise a plurality of polymer arms linked to a core region, where the polymer arms comprise a hydrophilic polymer segment. One end of the hydrophilic polymer segment is covalently attached to the core region through a suitable linkage, and a first reactive moiety is covalently attached to an opposite end of the hydrophilic polymer segment through a suitable linkage.

Reactive multi-arm polymers for use in the present disclosure include polymers having from 3 to 100 arms, for example ranging anywhere from 3 to 4 to 5 to 6 to 7 to 8 to 9 to 10 to 11 to 12 to 15 to 20 to 25 to 50 to 75 to 100 arms (in other words, having a number of arms ranging between any two of the preceding values).

Reactive end groups include those that comprise electrophilic groups. Electrophilic groups may be selected, for example, from cyclic imide ester groups, such as succinimide ester groups,

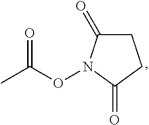

maleimide ester groups, glutarimide ester groups, diglycolimide ester groups, phthalimide ester groups, and bicyclo[2.2.1]hept-5-ene-2,3-dicarboxylic acid imide ester groups,

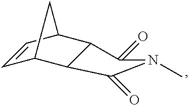

imidazole ester groups, imidazole carboxylate groups and benzotriazole ester groups, among other possibilities.

The electrophilic groups may be linked to the hydrophilic polymer segment and the hydrophilic polymer segment may be linked to the core through any suitable linking moiety, which may be selected, for example, from a bond, a linking moiety that comprises an alkyl group, a linking moiety that comprises an ether group, a linking moiety that comprises an ester group, a linking moiety that comprises an amide group, a linking moiety that comprises an amine group, a linking moiety that comprises a carbonate group, a linking moiety that comprises a urethane group, a linking moiety that comprises a urea group, a linking moiety that comprises a ketone group, or a linking moiety that comprises a combination of two or more of any of the foregoing groups, among others. In some embodiments, the linking moiety comprises a hydrolysable ester group. In some embodiments, the linking moiety comprises a hydrolysis-resistant amide group.

Hydrophilic polymer segments can be selected from any of a variety of synthetic, natural, or hybrid synthetic-natural hydrophilic polymer segments. Examples of hydrophilic polymer segments include those that are formed from one or more hydrophilic monomers. Hydrophilic monomers may be selected, for example, from the following monomers: C₁-C₆-alkylene oxide monomers (e.g., ethylene oxide, propylene oxide, tetramethylene oxide, etc.), polar aprotic vinyl monomers (e.g. N-vinyl pyrrolidone, acrylamide, N-methyl acrylamide, dimethyl acrylamide, N-vinyl imidazole, 4-vinylimidazole, sodium 4-vinylbenzenesulfonate, etc.), dioxanone, ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, N-isopropylacrylamide, amino acids and sugars.

Hydrophilic polymer segments may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, poly(N-isopropylacrylamide) segments, polypeptide segments, and polysaccharide segments. Polysaccharide segments include those that contain one or more uronic acid species, such as galacturonic acid, glucuronic acid and/or iduronic acid, with particular examples of polysaccharide segments including alginic acid, hyaluronic acid, pectin, agaropectin, carrageenan, gellan gum, gum arabic, guar gum, xanthan gum, and carboxymethyl cellulose moieties.

Polymer segments for use in the multi-arm polymers of the present disclosure typically contain from 10 monomer units or less to 1000 monomer units or more, for example, ranging anywhere from 5 to 10 to 20 to 50 to 100 to 200 to 500 to 1000 to 2000 monomer units.

In certain embodiments, the core region comprises a residue of a polyhydroxy compound comprising three or more hydroxyl groups, also referred to herein as a “polyol”, which is used to form the polymer arms. In certain beneficial embodiments, the core region comprises a residue of a polyol that contains from 3 to 100 hydroxyl groups.

Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, dipentaerythritol, tripentaerythritol, adonitol, hexaglycerol, dulcitol, erythrose, threose, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, nonamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl) ethane, and 2,6-bis(hydroxyalkyl) cresols, among others.

Illustrative polyols also include polyhydroxylated polymers. For example, in some embodiments, the core region comprises a polyhydroxylated polymer residue such as a poly(vinyl alcohol) residue, poly(allyl alcohol), polyhydroxyethyl acrylate residue, or a polyhydroxyethyl methacrylate residue, among others. Such polyhydroxylated polymer residues may range, for example, from 3 to 100 monomer units in length.

In some embodiments, the multi-arm polymers of the present disclosure may be radiopaque. One way to introduce radiopacity to the multi-arm polymers is to employ an iodine-containing polyol when forming the multi-arm polymers. Illustrative iodinated polyols include iodine-containing polyols that are known for use as iodinated contrast agents, such as 1,3,5-triiodo-2,4,6-tris-hydroxymethylbenzene, iodixanol, iotrolan, iohexol, ioversol, iopamidol, iohexol impurity J, and iopromide, among others.

Reactive multi-arm polymers for use in the present disclosure can be formed from hydroxy-terminated multi-arm polymers having arms that comprise one or more hydroxyl end groups. In some embodiments of the present disclosure, a polyol such as one of those described below, among others, may be used as multi-functional initiator for polymer chain growth. For example, the polyol may be used as an initiator for ring-opening polymerization of ethylene oxide to form polyethylene oxide (PEO) segments (also referred to a polyethylene glycol, or PEG, segments) at each of the hydroxyl groups of the polyol. The resulting hydroxyl-terminated PEG segments possess tunable hydrophilicity depending on the desired water-solubility of the resulting multi-arm polymer, for example, with increasing PEG segment length leading to increasing hydrophilicity. Hydroxyl-terminated multi-arm polymers are also available commercially. For example, hydroxyl-terminated four-arm PEG, hydroxyl-terminated six-arm PEG, and hydroxyl-terminated eight-arm PEG are available from JenKem Technology USA, Plano, TX, USA.

In some embodiments, a hydroxy-terminated multi-arm hydrophilic polymer may be reacted with a cyclic anhydride to form carboxylic-acid-terminated polymer in which carboxylic acid end groups are linked to hydrophilic polymer segments through hydrolysable ester groups. For example, terminal hydroxyl groups of the hydrophilic polymer segments may be reacted with a cyclic anhydride (e.g., glutaric anhydride, succinic anhydride, malonic anhydride, adipic anhydride, diglycolic anhydride, etc.) to form a carboxylic-acid-terminated segment such as a glutaric-acid-terminated segment, a succinic-acid-terminated segment, a malonic-acid-terminated segment, an adipic-acid-terminated segment, a diglycolic-acid-terminated segment, and so forth.

The preceding cyclic anhydrides, among others, may be reacted with a hydroxy-terminated multi-arm hydrophilic polymer under basic conditions to form a carboxylic-acid-terminated multi-arm hydrophilic polymer comprising carboxylic acid end groups that are linked to hydrophilic polymer segments through hydrolysable ester group.

In some embodiments, an amino-terminated multi-arm hydrophilic polymer may be reacted with a cyclic anhydride to form carboxylic-acid-terminated polymer in which carboxylic acid end groups are linked to hydrophilic polymer segments through hydrolysis-resistant amide groups.

Amino-terminated multi-arm hydrophilic polymers that comprise a core region and a plurality of polymer arms with amino end groups may be formed, for example, by reacting a multi-arm polymer that comprises a core region and a plurality of polymer arms with hydroxyalkyl end groups with methanesulfonyl chloride to form a multi-arm polymer that comprises a core region and a plurality of polymer arms with methanesulfonate end groups. The methanesulfonate end groups may then be reacted with ammonia to form a multi-arm polymer that comprises a core region and a plurality of polymer arms with amino end groups. Amino-terminated multi-arm polymers are also available commercially. For example, primary-amine-terminated four-arm PEG, primary-amine-terminated six-arm PEG and primary-amine-terminated eight-arm PEG are available from JenKem Technology USA.

In some embodiments, terminal amino groups of amino-terminated hydrophilic polymer segments may be reacted with a cyclic anhydride (e.g., glutaric anhydride, succinic anhydride, malonic anhydride, adipic anhydride, diglycolic anhydride, etc.) to form carboxylic-acid-terminated hydrophilic polymer segments (e.g., glutaramide-terminated segments, succinamide-terminated segments, malonamide-terminated segments, adipamide-terminated segments, diglycolamide-terminated segments, etc.) that comprise a carboxylic acid end group that is linked to a hydrophilic polymer segment through a hydrolysis-resistant amide group.

Carboxylic-acid-terminated multi-arm polymers are also available commercially. For example, carboxylic-acid-terminated four-arm PEG and carboxylic-acid-terminated eight-arm PEG (without hydrolysable ester groups) are available from JenKem Technology USA.

An electrophilic moiety, such as a cyclic-imide-containing moiety, may be linked to the carboxylic-acid-terminated multi-arm hydrophilic polymer. For instance, an N-hydroxy cyclic imide compound (e.g., N-hydroxysuccinimide, N-hydroxymaleimide, N-hydroxyglutarimide, N-hydroxyphthalimide, or N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic-acid-terminated multi-arm hydrophilic polymer in the presence of a suitable coupling agent (e.g., one of those described above) form an activated ester group, in particular, a cyclic imide ester group (e.g., an succinimide ester group, an maleimide ester group, an glutarimide ester group, an phthalimide ester group, a diglycolimide ester group, bicyclo[2.2.1]hept-5-ene-2,3-dicarboxylic acid imide ester group, etc.) that is linked to a hydrophilic polymer segment.

Where the carboxylic-acid-terminated multi-arm hydrophilic polymer comprises a hydrolysable ester group, various reactive diester groups can be formed. For example, in the particular case of N-hydroxysuccinimide as an N-hydroxy cyclic imide compound, exemplary reactive end groups include succinimidyl malonate groups, succinimidyl glutarate groups, succinimidyl succinate groups, succinimidyl adipate groups, and succinimidyl diglycolate groups, among others. In the particular case of HONB as an N-hydroxy cyclic imide compound, exemplary reactive end groups include bicyclo[2.2.1]hept-5-ene-2,3-dicarboxylic acid imidyl malonate groups, bicyclo[2.2.1]hept-5-ene-2,3-dicarboxylic acid imidyl glutarate groups, bicyclo[2.2.1]hept-5-ene-2,3-dicarboxylic acid imidyl succinate groups, bicyclo[2.2.1]hept-5-ene-2,3-dicarboxylic acid imidyl adipate groups, and bicyclo[2.2.1]hept-5-ene-2,3-dicarboxylic acid imidyl diglycolate groups, among others. In the particular case of N-hydroxymaleimide as an N-hydroxy cyclic imide compound, exemplary reactive end groups include maleimidyl malonate groups, maleimidyl glutarate groups, maleimidyl succinate groups, maleimidyl adipate groups, and maleimidyl diglycolate groups, among others. In the particular case of N-hydroxyglutarimide as an N-hydroxy cyclic imide compound, exemplary reactive end groups include glutarimidyl malonate groups, glutarimidyl glutarate groups, glutarimidyl succinate groups, glutarimidyl adipate groups, glutarimidyl diglycolate groups, among others. In the particular case of N-hydroxyphthalimide as an N-hydroxy cyclic imide compound, exemplary reactive end groups include phthalimidyl malonate groups, phthalimidyl glutarate groups, phthalimidyl succinate groups, phthalimidyl adipate groups, and phthalimidyl diglycolate groups, among others.

Where the carboxylic-acid-terminated multi-arm hydrophilic polymer comprises a hydrolysis-resistant amine group, various reactive ester-amide groups can be formed. For example, in the particular case of N-hydroxysuccinimide as an N-hydroxy cyclic imide compound, exemplary reactive end groups include succinimidyl malonamide groups, succinimidyl glutaramide groups, succinimidyl succinamide groups, succinimidyl adipamide groups, and succinimidyl diglycolamide groups, among others. In the particular case of HONB as an N-hydroxy cyclic imide compound, exemplary reactive end groups include bicyclo[2.2.1]hept-5-ene-2,3-dicarboxylic acid imidyl malonamide groups, bicyclo[2.2.1]hept-5-ene-2,3-dicarboxylic acid imidyl glutaramide groups, bicyclo[2.2.1]hept-5-ene-2,3-dicarboxylic acid imidyl succinamide groups, bicyclo[2.2.1]hept-5-ene-2,3-dicarboxylic acid imidyl adipamide groups, and bicyclo[2.2.1]hept-5-ene-2,3-dicarboxylic acid imidyl diglycolamide groups, among others. In the particular case of N-hydroxymaleimide as an N-hydroxy cyclic imide compound, exemplary reactive end groups include maleimidyl malonamide groups, maleimidyl glutaramide groups, maleimidyl succinamide groups, maleimidyl adipamide groups, and maleimidyl diglycolamide groups, among others. In the particular case of N-hydroxyglutarimide as an N-hydroxy cyclic imide compound, exemplary reactive end groups include glutarimidyl malonamide groups, glutarimidyl glutaramide groups, glutarimidyl succinamide groups, glutarimidyl adipamide groups, glutarimidyl diglycolamide groups, among others. In the particular case of N-hydroxyphthalimide as an N-hydroxy cyclic imide compound, exemplary reactive end groups include phthalimidyl malonamide groups, phthalimidyl glutaramide groups, phthalimidyl succinamide groups, phthalimidyl adipamide groups, and phthalimidyl diglycolamide groups, among others.

Some succinimide-terminated multi-arm polymers are also available commercially. For example, for hydrolysable applications, succinimidyl-glutarate-terminated four-arm PEG and succinimidyl-glutarate-terminated eight-arm PEG are available from JenKem Technology USA. For hydrolysis-resistant applications, succinimidyl-glutaramide-terminated four-arm PEG is available from JenKem Technology USA, and succinimidyl-glutaramide-terminated eight-arm PEG is available from Advanced BioChemicals, Lawrenceville, GA, USA.

In some aspects of the present disclosure, systems are provided that are configured to deliver (a) a cell-adhesive polyamino compound in accordance with the present disclosure and (b) a reactive multi-arm polymer as described herein. The cell-adhesive polyamino compound and the reactive multi-arm polymer are combined under conditions such that the amino groups of the cell-adhesive polyamino compound and the reactive end groups of the reactive multi-arm polymer crosslink with one another. In certain embodiments, those conditions comprise an environment having a basic pH, for example, a pH ranging from about 8.5 to about 12, a pH ranging from about 9 to about 11, or a pH ranging from about 9.5 to about 10.5. Such systems can be used to form crosslinked hydrogels, either in vivo or ex vivo.

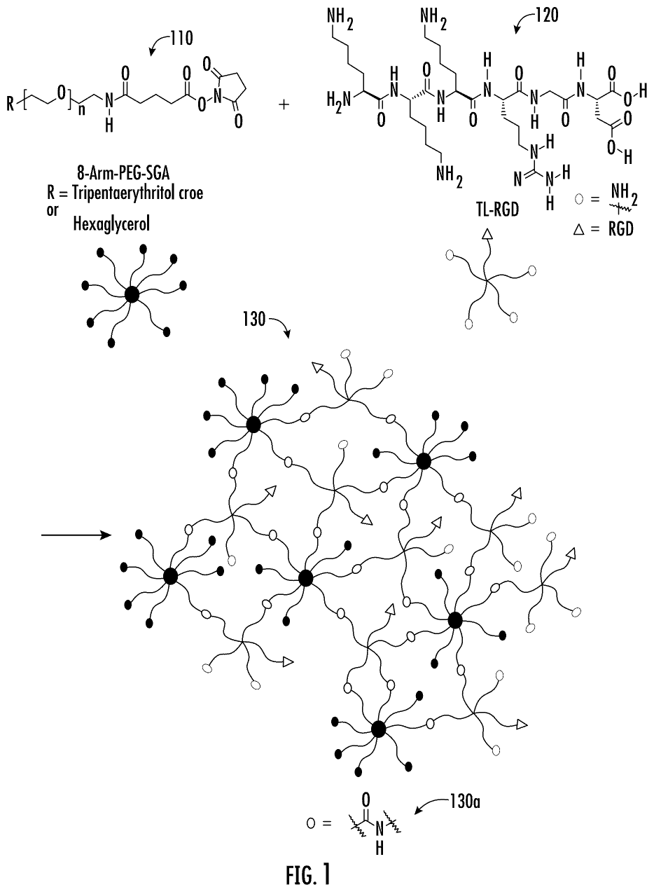

A particular example of a crosslinking reaction is schematically illustrated in FIG. 1, which shows a covalent crosslinking reaction between (a) cyclic amide ester groups of a reactive multi-arm polymer, specifically, a multi-arm polymer 110 comprising eight polyethylene glycol arms where n is an integer, each arm extending from a polyol residue core R, which may be, for example, a tripentaerythritol residue or a hexaglycerol residue, and each terminating with a succinimidyl glutaramide group (8-armPEG-SGA) and (b) reactive amino groups of a cell-adhesive polyamino compound, specifically, Lys-Lys-Lys-Arg-Gly-Asp 120 as described herein, whereby amide crosslinking groups 130a are formed, resulting in a crosslinked hydrogel 130. The crosslinking reaction shown is inhibited at acidic pH, but occurs spontaneously at basic pH.

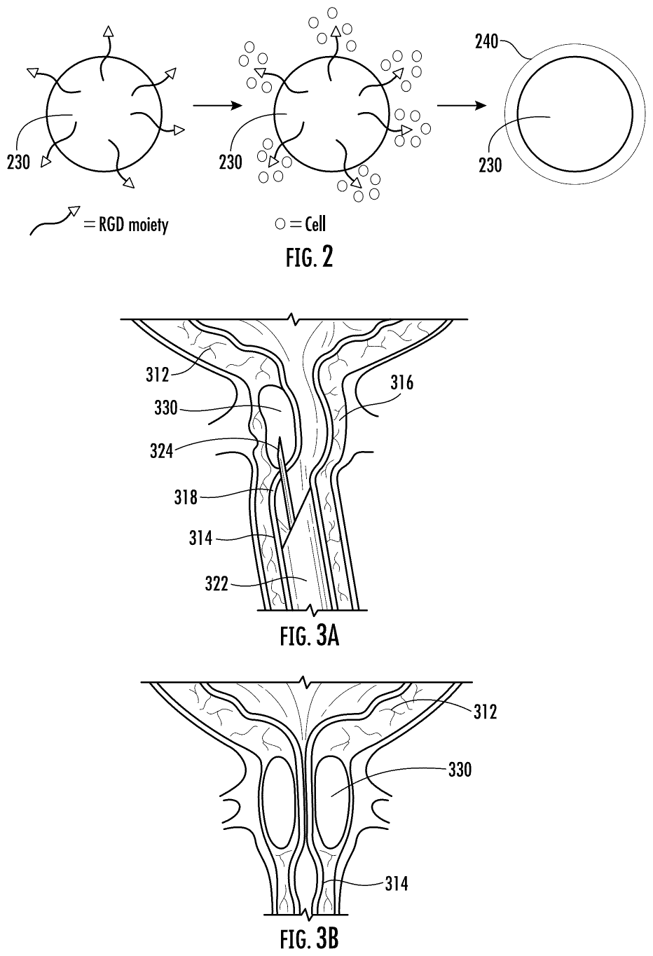

As illustrated schematically in FIG. 2, because the crosslinked hydrogel implant 230 contains unreacted RGD moieties originating from the cell-adhesive polyamino compound, upon implantation, the RGD moieties can act as a template to induce cell adhesion and subsequent cell proliferation as shown in the center of FIG. 2. Ultimately, the cell proliferation may result in growth of tissue 220 that surrounds the crosslinked hydrogel implant 230, which can resist migration of the crosslinked hydrogel implant 230 from the implantation site.

In some aspects of the present disclosure, a system is provided that comprises (a) a first composition that comprises a cell-adhesive polyamino compound as described herein and (b) a second composition that comprises a reactive multi-arm polymer as described herein.

The first composition may be a first fluid composition comprising the cell-adhesive polyamino compound or a first dry composition that comprises the cell-adhesive polyamino compound, to which a suitable fluid such as water for injection, saline, etc. can be added to form a first fluid composition. In addition to the cell-adhesive polyamino compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive multi-arm polymer or a second dry composition that comprises the reactive multi-arm polymer, to which a suitable fluid such as water for injection, saline, etc. can be added to form a second fluid composition. In addition to the reactive multi-arm polymer, the second composition may further comprise additional agents, including therapeutic agents, imaging agents, colorants, tonicity adjusting agents, suspension agents, wetting agents, and pH adjusting agents as described below.

In some embodiments, the cell-adhesive polyamino compound is initially combined with the reactive multi-arm polymer under conditions where crosslinking between the electrophilic moieties of the reactive multi-arm polymer and the amino groups of the cell-adhesive polyamino compound is suppressed (e.g., an acidic pH, in some embodiments). Then, when crosslinking is desired, the conditions are changed such that crosslinking is increased (e.g., a change from an acidic pH to a basic pH, in some embodiments), leading to crosslinking between same, thereby forming the crosslinked product.

In particular embodiments, the system comprises (a) a first composition that comprises a cell-adhesive polyamino compound as described hereinabove, (b) a second composition that comprises a reactive multi-arm polymer as described hereinabove, and (c) a third composition, specifically, an accelerant composition, that contains an accelerant that is configured to accelerate a crosslinking reaction between the cell-adhesive polyamino compound and the reactive multi-arm polymer.

The first composition may be a first fluid composition comprising the cell-adhesive polyamino compound that is buffered to an acidic pH or a first dry composition that comprises the cell-adhesive polyamino compound and acidic buffering composition, to which a suitable fluid such as water for injection, saline, etc. can be added to form a first fluid composition comprising the cell-adhesive polyamino compound that is buffered to an acidic pH. In some embodiments, for example, the acidic buffering composition may comprise monobasic sodium phosphate, among other possibilities. The first fluid composition comprising the cell-adhesive polyamino compound may have a pH ranging, for example, from about 3 to about 6.5. In addition to the cell-adhesive polyamino compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive multi-arm polymer or a second dry composition that comprises the reactive multi-arm polymer from which a fluid composition is formed, for example, by the addition of a suitable fluid such as water for injection, saline, or the first fluid composition comprising the cell-adhesive polyamino compound that is buffered to an acidic pH. In addition to the reactive multi-arm polymer, the second composition may further comprise additional agents, including therapeutic agents, imaging agents, colorants, tonicity adjusting agents, suspension agents, wetting agents, and pH adjusting agents as described below.

In a particular embodiment, the first composition is a first fluid composition comprising the cell-adhesive polyamino compound that is buffered to an acidic pH and the second composition comprises a dry composition that comprises the reactive multi-arm polymer. The first composition may then be mixed with the second composition to provide a prepared fluid composition that is buffered to an acidic pH and comprises the cell-adhesive polyamino compound and the reactive multi-arm polymer. In a particular example, a syringe may be provided that contains the first fluid composition comprising the cell-adhesive polyamino compound that is buffered to an acidic pH, and a vial may be provided that comprises the dry composition (e.g., a powder) that comprises the reactive multi-arm polymer. The syringe may then be used to inject the first fluid composition into the vial containing the reactive multi-arm polymer to form a prepared fluid composition that contains the cell-adhesive polyamino compound and the reactive multi-arm polymer, which can be withdrawn back into the syringe for administration.

The third composition may be a fluid accelerant composition that is buffered to a basic pH or a dry composition that comprises a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 8.5 to about 11.5. In addition to the above, the fluid accelerant composition may further comprise additional agents, including those described below.

A prepared fluid composition that is buffered to an acidic pH and comprises the cell-adhesive polyamino compound and the reactive multi-arm polymer as described above (as well as additional agents in some cases), and a fluid accelerant composition that is buffered to basic pH as described above (which may include additional agents in some cases), may be combined form crosslinked hydrogels, either in vivo or ex vivo.

In some particular embodiments of the present disclosure, a kit is provided that include a first reservoir (e.g., a vial or syringe barrel) containing a first composition comprising a cell-adhesive polyamino compound as described herein and an acidic buffer, a second reservoir (e.g., a vial or syringe barrel) containing a second composition comprising containing a reactive multi-arm polymer as described herein, a third reservoir (e.g., a vial or syringe barrel) containing a third composition comprising a buffered accelerant as described herein, additional apparatus, as required, for combining the first and second compositions to provide a prepared fluid composition that is buffered to an acidic pH and comprises the cell-adhesive polyamino compound and the reactive multi-arm polymer, and additional apparatus for combining and delivering the prepared fluid composition and buffered accelerant to a patient.

In more particular embodiments, the kit may comprise a vial containing a reactive multi-arm polymer as described herein in dry (e.g., powdered) form, a first syringe containing a fluid composition comprising a cell-adhesive polyamino compound as described herein that is buffered to an acidic pH, a second syringe containing a buffered accelerant solution as described herein, a needle and/or tube, a Y-connector, a syringe holder, a plunger cap and a vial adapter. Such components may be placed in sterile packaging, for example, in one or more packaged sterile trays.

The compositions described herein may be sterilized using any suitable method. For example, the compositions may be autoclaved while inside a reservoir, such as a syringe barrel, vial, or ampule by heating the mixture at or to a temperature of about 121° C. Alternatively or additionally, the compositions may be sterilized via sterile filtration and/or by supercritical CO2, gamma, x-ray or electron beam irradiation.

Additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly (DP-ribose) polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxylic acid groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents (beyond the radiopaque iodine atoms that may be present in the cell-adhesive polyamino compound and/or reactive multi-arm polymer) such as metallic particles, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

A prepared fluid composition that is buffered to an acidic pH and comprises the cell-adhesive polyamino compound and the reactive multi-arm polymer as described above, and a fluid accelerant composition that is buffered to basic pH as described above, may be combined form crosslinked hydrogels, either in vivo or ex vivo.

In various embodiments, a system is provided that includes one or more delivery devices for delivering first and second compositions to a subject.

In some embodiments, the system may include a delivery device that comprises a first reservoir that contains a first composition that comprises a cell-adhesive polyamino compound as described above and a second reservoir that contains a second composition that comprises a reactive multi-arm polymer that comprises a plurality of electrophilic moieties that are reactive with the amino moieties of the cell-adhesive polyamino compound as described above.

In some embodiments, the system may include a delivery device that comprises a first reservoir that contains a first composition that comprises the cell-adhesive polyamino compound and the reactive multi-arm polymer and is buffered to an acidic pH, such as the prepared fluid composition previously described, and a second reservoir that contains second composition, such as the fluid accelerant composition previously described.

In either case, during operation, the first composition and second composition are dispensed from the first and second reservoirs and combined, whereupon the cell-adhesive polyamino compound and the reactive multi-arm polymer and crosslink with one another to form a crosslinked hydrogel.

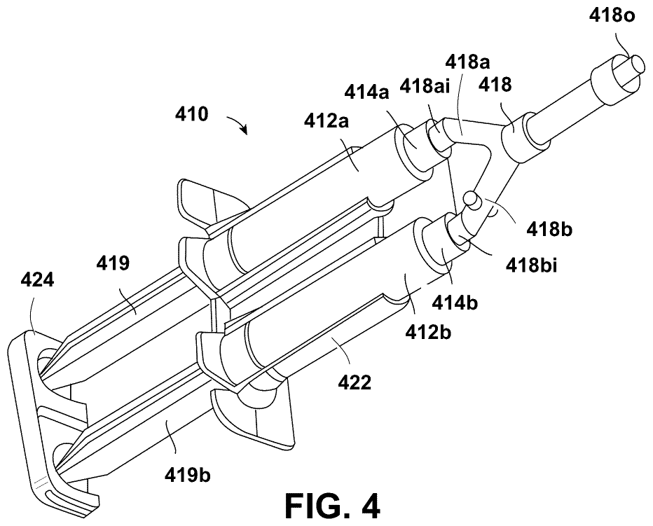

In particular embodiments, and with reference to FIG. 4, the system may include a delivery device 410 that comprises a double-barrel syringe, which includes a first barrel 412a having a first barrel outlet 414a, which first barrel contains a first fluid composition as described above, a first plunger 419a that is movable in the first barrel 412a, a second barrel 412b having a second barrel outlet 414b, which second barrel 412b contains a second fluid composition as described above, and a second plunger 419b that is movable in the second barrel 412b. In some embodiments, the device 410 may further comprise a mixing section 418 (e.g., a Y-connector) having a first mixing section inlet 418ai in fluid communication with the first barrel outlet 414a, a second mixing section inlet 418bi in fluid communication with the second barrel outlet 414b, and a mixing section outlet 4180. Also shown are a syringe holder 422 configured to hold the first and second syringe barrels 412a, 412b, in a fixed relationship and a plunger cap 424 configured to hold the first and second plungers 419a, 419b in a fixed relationship.

In some embodiments, the delivery device may further comprise a needle and/or flexible catheter that is configured to receive the first and second fluid compositions from the first and second barrels. For example, a needle or catheter tube may be configured to form a fluid connection with an outlet of a mixing section by attaching the cannula or catheter tube to an outlet of the mixing section, for example, via a suitable fluid connector such as a Luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions mix and ultimately crosslink to form a crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, in some embodiments, the first and second fluid compositions or a fluid admixture thereof can be injected as a bulking agent. For example, the first and second fluid compositions or a fluid admixture thereof may be injected to increase coaptation of a bodily sphincter, such as an anal sphincter for fecal incontinence or a urinary sphincter.

In other examples, the first and second fluid compositions or a fluid admixture thereof can be injected as a bulking agent into tissue around the ureteral orifices for the treatment of vesicoureteral reflux, the first and second fluid compositions or a fluid admixture thereof can be injected for tissue augmentation or regeneration, including cosmetic tissue augmentation, the first and second fluid compositions or a fluid admixture thereof can be injected to provide spacing between tissues, the first and second fluid compositions or a fluid admixture thereof can be injected (e.g., in the form of blebs) to provide fiducial markers or organ marking, the first and second fluid compositions or a fluid admixture thereof can be injected for tissue augmentation or regeneration, including cosmetic tissue augmentation, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, the first and second fluid compositions or a fluid admixture thereof can be injected for seminal vesicle occlusion, the first and second fluid compositions or a fluid admixture thereof can be injected as lifting agents for internal cyst removal, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure or injected for closure of an atrial septal defect. In some embodiments, the first and second fluid compositions or a fluid admixture thereof may be injected into the left atrial appendage after the introduction of a closure device such as the Watchman® left atrial appendage closure device available from Boston Scientific Corporation.

After administration of the compositions of the present disclosure (either separately as first and second fluid compositions that mix in vivo or as a fluid admixture of the first and second fluid compositions) a crosslinked hydrogel is ultimately formed at the administration location.

During and/or after administration, the compositions of the present disclosure can be imaged using a suitable imaging technique. Typically, the imaging technique is an x-ray-based imaging technique, such as computerized tomography or X-ray fluoroscopy, or a near near-IR fluorescence spectrometry-based technique.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant an embolic composition comprising a crosslinked product of the first and second fluid compositions, a procedure to implant a composition comprising a crosslinked product of the first and second fluid compositions to provide seminal vessel occlusion, a procedure to implant a lifting agent comprising a crosslinked product of the first and second fluid compositions, a procedure to introduce a left atrial appendage closure composition comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, injection for closure of an atrial septal defect, injection for seminal vessel occlusion, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, crosslinked hydrogels may be in any desired form, including a slab, a cylinder, a coating, or a particle. In some embodiments, the crosslinked hydrogel is dried and then granulated into particles of suitable size. Granulating may be by any suitable process, for instance by grinding (including cryogrinding), homogenization, crushing, milling, pounding, or the like. Sieving or other known techniques can be used to classify and fractionate the particles. Crosslinked hydrogel particles formed using the above and other techniques may varying widely in size, for example, having an average size ranging from 50 to 950 microns.

In addition to a crosslinked hydrogel as described above, crosslinked hydrogel compositions in accordance with the present disclosure may contain additional agents, including therapeutic agents, imaging agents, colorants, tonicity adjusting agents, suspension agents, wetting agents, and pH adjusting agents as described above.

The crosslinked hydrogel compositions of the present disclosure may be sterilized using any suitable method. For example, the compositions may be autoclaved while inside a reservoir, such as a syringe barrel, vial, or ampule by heating the mixture at or to a temperature of about 121° C. Alternatively or additionally, the compositions may be sterilized via sterile filtration and/or by supercritical CO2, gamma, x-ray or electron beam irradiation.

In various embodiments, kits are provided that include one or more delivery devices for delivering the crosslinked hydrogel to a subject. Such systems may include one or more of the following: a syringe barrel, which may or may not contain a crosslinked hydrogel as described herein; a vial, which may or may not contain a crosslinked hydrogel as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the crosslinked hydrogel may be provided in dry form (e.g., powder form) or in a form that is ready for injection, such as an injectable hydrogel form (e.g., a suspension of crosslinked hydrogel particles).

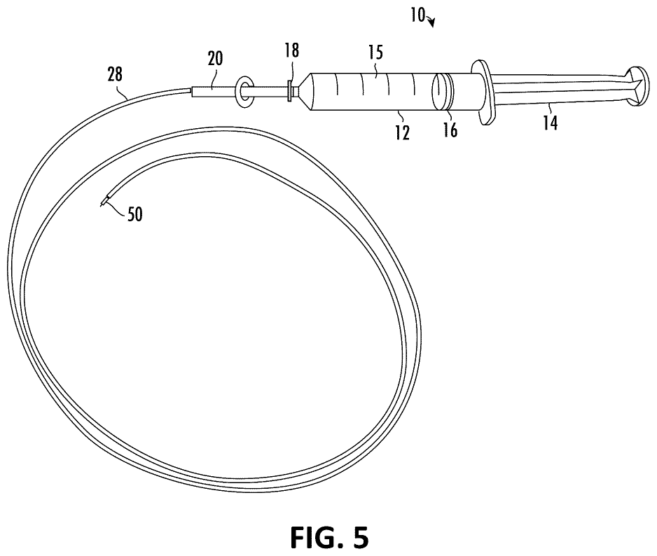

FIG. 5 illustrates a syringe 10 providing a reservoir for a crosslinked hydrogel compositions as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 28. The proximal end of the catheter 28 may include a suitable connection 20 for receiving the barrel 12. In other examples, the barrel 12 may be directly coupled to the injection needle 50. The syringe barrel 12 may serve as a reservoir, containing a crosslinked hydrogel composition 15 for injection through the needle 50.

The crosslinked hydrogel compositions described herein can be used for a number of purposes.

For example, crosslinked hydrogel compositions can be injected to provide spacing between tissues, crosslinked hydrogel compositions can be injected (e.g., in the form of blebs) to provide fiducial markers, crosslinked hydrogel compositions can be injected for tissue augmentation or regeneration, crosslinked hydrogel compositions can be injected as a filler or replacement for soft tissue, crosslinked hydrogel compositions can be injected to provide mechanical support for compromised tissue, crosslinked hydrogel compositions be injected as a scaffold, and/or crosslinked hydrogel compositions can be injected as a carrier of therapeutic agents in the treatment of diseases and cancers and the repair and regeneration of tissue, among other uses.

During and/or after administration, the crosslinked hydrogel compositions of the present disclosure can be imaged using a suitable imaging technique.

As seen from the above, the crosslinked hydrogel compositions of the present disclosure may be used in a variety of medical procedures, including the following, among others: a procedure to implant a fiducial marker comprising a crosslinked hydrogel, a procedure to implant a tissue regeneration scaffold comprising a crosslinked hydrogel, a procedure to implant a tissue support comprising a crosslinked hydrogel, a procedure to implant a tissue bulking agent comprising a crosslinked hydrogel, a procedure to implant a therapeutic-agent-containing depot comprising a crosslinked hydrogel, a tissue augmentation procedure comprising implanting a crosslinked hydrogel, a procedure to introduce a crosslinked hydrogel between a first tissue and a second tissue to space the first tissue from the second tissue.

The crosslinked hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, injection for seminal vessel occlusion, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

By way of illustration, a particular medical procedure will now be described, which pertains to stress urinary incontinence. Many post-menopausal women complain of symptoms relating to stress urinary incontinence. Recent innovations with long-term implantable hydrogels that are delivered into the urethral wall have caught medical attention for several reasons including the fact that the procedure is fast, relatively non-invasive, and can be repeated at a later time, and there is a low risk of risk of excessive inflammatory response, such as tissue growth complication.

The procedure is illustrated in FIGS. 3A and 3B, which show a portion of the human anatomy that includes a lower portion of the bladder wall 312, an upper portion of the urethra 314, and the bladder neck 316, which is a group of muscles that connect the bladder to the urethra. A fluid admixture of first and second fluid compositions as described herein, or a crosslinked hydrogel composition as described herein is injected by a transurethral route using a suitable delivery device such as those described here. For example, a fluid admixture of first and second fluid compositions may be delivered using a device like that shown in FIG. 4, where a flexible catheter (not shown) and needle 324 are attached to the mixing section outlet 4180 of the delivery device 1010, or a crosslinked hydrogel composition may be delivered using a device like that shown in FIG. 5, where a flexible catheter (not shown) and needle 324 are attached to the syringe barrel 15 of the device 10. In the embodiment shown, the injection is performed with the aid of a cystoscope 322 through the urethral wall 318 into the bladder neck 316, increasing coaptation of the urinary sphincter. In cases where a fluid admixture of first and second fluid compositions is administered, the polyamino compound and the reactive polymer in the fluid admixture crosslink after injection to form a hydrogel 330. In cases where a crosslinked hydrogel composition is administered, a hydrogel 330 (e.g., in the form of a particle suspension) is injected directly. Because the hydrogel 330 includes cell-adhesive peptide moieties, cell adhesion and tissue growth are promoted upon implantation, which in turn promote resistance to migration of the hydrogel 330. Typically, a biostable implant is desired, and the multi-arm polymer used to form the hydrogel 330 does not contain any hydrolysable groups. However, where a biodegradable implant is desired, the multi-arm polymer used to form the hydrogel 330 may include hydrolysable groups.

1. A system for forming a hydrogel that comprises a cell-adhesive polyamino compound and a reactive multi-arm polymer that comprises a plurality of hydrophilic polymer arms having reactive end groups that are reactive with amino groups of the cell-adhesive polyamino compound.

2. The system of claim 1, wherein the cell-adhesive polyamino compound comprises a polyamino moiety that is linked to a cell-adhesive peptide moiety by an amide group.

3. The system of claim 2, wherein the polyamino moiety comprises a plurality of —(CH₂)ₓ—NH₂ groups where x is 0, 1, 2 3, 4, 5 or 6.

4. The system of claim 2, wherein the polyamino moiety is a polyamino peptide moiety.

5. The system of claim 2, wherein the polyamino moiety comprises two or more amino acid residues selected from residues of lysine, ornithine, and combinations thereof.

6. The system of claim 1, wherein the cell-adhesive peptide moiety comprises one or more Arg-Gly-Asp sequences.

7. The system of claim 1, wherein the cell-adhesive polyamino compound comprises a covalently attached iodine atom.

8. The system of claim 1, wherein the polyamino moiety comprises a residue of a carboxylic-acid-containing iodinated molecule.

9. The system of claim 1, wherein the cell-adhesive polyamino compound comprises an iodinated amino acid residue.

10. The system of claim 1, wherein the hydrophilic polymer arms comprise polymer segments that comprise one or more hydrophilic monomers selected from ethylene oxide, N-vinyl pyrrolidone, oxazoline monomers, hydroxyethyl acrylate, and hydroxyethyl methacrylate.

11. The system of claim 1, wherein the reactive end groups are linked to the hydrophilic polymer arms by a hydrolysable ester.

12. The system of claim 1, wherein the reactive multi-arm polymer does not comprise a hydrolysable ester group.

13. The system of claim 1, wherein the reactive end groups are electrophilic groups.

14. The system of claim 1, wherein the system comprises a first composition that comprises the cell-adhesive polyamino compound, a second composition that comprises the reactive multi-arm polymer, and an optional accelerant composition.

15. The system of claim 14, wherein the first composition is provided in a syringe barrel, the second composition is provided in a vial, and the accelerant composition is provided in a syringe barrel.

16. A crosslinked product of the cell-adhesive polyamino compound and the reactive multi-arm polymer of the system of claim 1.

17. The crosslinked product of claim 16, wherein the crosslinked product does not contain hydrolysable ester groups.

18. An injectable suspension comprising particles of the crosslinked product of claim 16.

19. A method of treatment comprising administering to a subject a mixture that comprises a cell-adhesive polyamino compound and a reactive multi-arm polymer that comprises a plurality of hydrophilic polymer arms having reactive end groups that are reactive with amino groups of the cell-adhesive polyamino compound under conditions such that the cell-adhesive polyamino compound and the reactive multi-arm polymer cross-link after administration.

20. A method of treatment comprising administering to a subject a crosslinked product of a cell-adhesive polyamino compound and a reactive multi-arm polymer that comprises a plurality of hydrophilic polymer arms having reactive end groups that are reactive with amino groups of the cell-adhesive polyamino compound.
